(19) United States
(12) Patent Application Publication
(10) Pub. No.: US 2017/0225036 A1
(43) Pub. Date: Aug. 10, 2017

(54) INFORMATION PROCESSING DEVICE, INFORMATION PROCESSING METHOD, AND PROGRAM

(71) Applicant: SONY CORPORATION, TOKYO (JP)

(72) Inventor: Masato KIMISHIMA, TOKYO (JP)

(21) Appl. No.: 15/519,401

(22) PCT Filed: Sep. 15, 2015
(86) PCT No.: PCT/JP2015/076127

§ 371 Date: Apr. 14, 2017

(30) Foreign Application Priority Data
Oct. 22, 2014 (JP) 2014-215033

(51) Int. Cl. A63B 24/00; A61B 5/0205; A61B 5/00; G01C 22/00

(52) U.S. Cl. CPC A63B 24/0062; G01C 22/006; A61B 5/0205; A61B 5/0022; A63B 24/0003; A61B 5/024

(57) ABSTRACT

[Object] To provide an information processing device capable of accurately calculating a moving distance on the basis of the number of steps. [Solution] The information processing device includes: a movement determination unit configured to determine whether or not a user is moving on the basis of at least one of a heart rate, a blood pressure, and a respiratory rate of the user; and a distance calculation unit configured to calculate a moving distance of the user on the basis of the number of steps of the user in a period in which the movement determination unit determines that the user is moving.

### TECHNICAL FIELD

The present disclosure relates to an information processing device, an information processing method, and a program.

### BACKGROUND ART

In recent years, there have increased people who take exercise such as running and walking on a daily basis for the purpose of health management or the like. Such people measure an amount of daily exercise and record the amount of daily exercise in the form of numerical data or the like in many cases in order to manage exercise conditions thereof.

Therefore, in order to meet a demand from the above people, a measurement instrument for detecting an exercise condition of a person fitted with the measurement instrument to measure the number of steps, a moving distance, calorie consumption, or the like is commercially available.

For example, Patent Literature 1 cited below discloses a pedometer for estimating an exercise load during walking, correcting the measured number of steps on the basis of the estimated exercise load, and outputting the number of steps.

### CITATION LIST

### Patent Literature

Patent Literature 1: JP 2010-165088A

### DISCLOSURE OF INVENTION

### Technical Problem

However, in a case where the pedometer disclosed in Patent Literature 1 cited above detects walking of a user, it is difficult to detect whether the walking is walking of the user with movement or walking of the user without movement, i.e., only stepping on the spot. Therefore, the measured number of steps includes the number of steps of walking of the user without movement, and thus it is difficult to calculate an accurate moving distance on the basis of the measured number of steps of the user.

In view of this, the present disclosure proposes an information processing device, an information processing method, and a program, each of which is new, improved, and capable of accurately calculating a moving distance on the basis of the number of steps.

### Solution to Problem

According to the present disclosure, there is provided an information processing device including: a movement determination unit configured to determine whether or not a user is moving on the basis of at least one of a heart rate, a blood pressure, and a respiratory rate of the user; and a distance calculation unit configured to calculate a moving distance of the user on the basis of the number of steps of the user in a period in which the movement determination unit determines that the user is moving.

According to the present disclosure, there is provided an information processing method including: determining whether or not a user is moving on the basis of at least one of a heart rate, a blood pressure, and a respiratory rate of the user; and calculating a moving distance of the user on the basis of the number of steps of the user in a period in which a central processing unit determines that the user is moving.

According to the present disclosure, there is provided a program causing a computer to function as: a movement determination unit configured to determine whether or not a user is moving on the basis of at least one of a heart rate, a blood pressure, and a respiratory rate of the user; and a distance calculation unit configured to calculate a moving distance of the user on the basis of the number of steps of the user in a period in which the movement determination unit determines that the user is moving.

According to the present disclosure, it is possible to determine whether or not a user is moving on the basis of biological information of a user such as a heart rate, a blood pressure, and a respiratory rate. Therefore, it is possible to exclude a period in which a user is not moving and calculate a moving distance of the user on the basis of the number of steps in a period in which the user is moving.

### Advantageous Effects of Invention

As described above, according to the present disclosure, it is possible to accurately calculate a moving distance of a user on the basis of the number of steps.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory view schematically showing an information processing device according to a first embodiment of the present disclosure.

FIG. 2 is a block diagram showing an internal configuration of the information processing device according to this embodiment.

FIG. 3 is a graph showing an example of a temporal change in heart rate obtained in a case where a user is running while stopping occasionally.

FIG. 4 is a graph showing an example of a temporal change in heart rate immediately after a user starts running.

FIG. 5 is a block diagram showing an internal configuration of an information processing device according to a modification example of this embodiment.

FIG. 6 is a flowchart showing an example of control operation executed by the information processing device according to this embodiment.

FIG. 7 is a block diagram showing an example of a hardware configuration of the information processing device according to this embodiment.

### MODE(S) FOR CARRYING OUT THE INVENTION

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

Note that description will be provided in the following order.
1. Information processing device according to embodiment of present disclosure
   1.1. Outline of information processing device
   1.2. Functional configuration of information processing device
   1.3. Modification example of information processing device
   1.4. Control of information processing device
2. Hardware configuration
3. Conclusion

### 1. INFORMATION PROCESSING DEVICE ACCORDING TO EMBODIMENT OF PRESENT DISCLOSURE

[1.1. Outline of Information Processing Device]

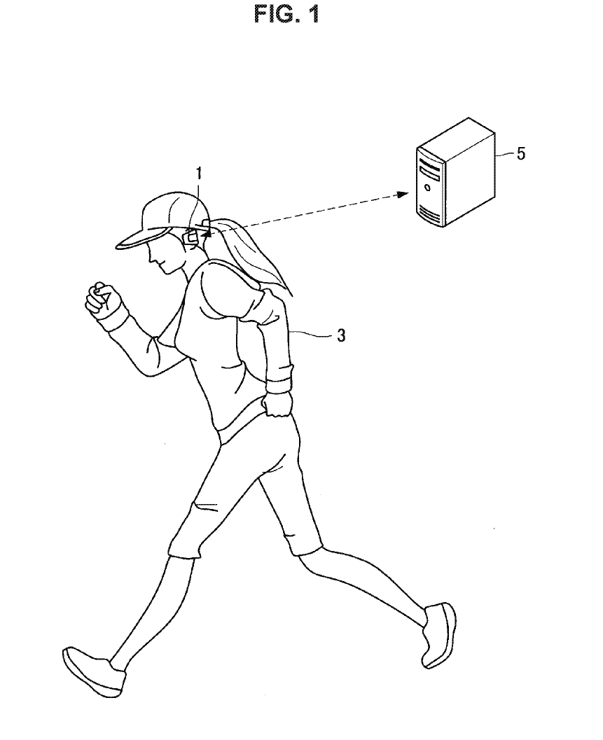

An outline of an information processing device according to an embodiment of the present disclosure will be described with reference to FIG. 1. FIG. 1 is an explanatory view schematically showing an information processing device according to the present embodiment.

As shown in FIG. 1, an information processing device 1 according to the present embodiment is mounted on, for example, a user 3 who runs or walks and detects walking of the user 3 with the use of various sensors and measures the number of steps. Further, the information processing device 1 acquires biological information of the user 3, such as a heart rate (substantially the same meaning as pulse rate), a blood pressure, and a respiratory rate, with the use of the various sensors.

Herein, a load applied to a body of the user 3 in a case where the user 3 is walking with movement (i.e., is running or walking) is larger than a load applied thereto in a case where the user 3 is walking without movement (i.e., is stepping on the spot). Therefore, the heart rate, the blood pressure, the respiratory rate, and the like of the user 3 obtained in a case where the user 3 is walking without movement are lower than those obtained in a case where the user 3 is walking with movement.

Therefore, by acquiring the biological information of the user 3 such as the heart rate, the blood pressure, and the respiratory rate with the use of the various sensors, the information processing device 1 can determine whether or not the user 3 is moving during walking on the basis of the acquired biological information of the user 3. With this, the information processing device 1 can calculate a moving distance of the user 3 on the basis of the number of steps in a period in which the information processing device 1 determines that the user 3 is moving, and therefore it is possible to accurately calculate the moving distance of the user 3.

Note that the biological information of the user 3 acquired by the various sensors is preferably a heart rate, a blood pressure, a respiratory rate, and the like which are biological information highly responsive to a load caused by physical exercise of the user 3. That is, the information processing device 1 can determine whether or not the user 3 is moving during walking with the use of at least one of the heart rate, the blood pressure, and the respiratory rate of the user 3.

Further, the information processing device 1 may communicate with a server device 5 via a network. For example, the information processing device 1 may transmit the measured number-of-step information and the acquired biological information of the user 3 to the server device 5 to cause the server device 5 to execute part of or the whole information processing to be executed in the information processing device 1. Herein, examples of the network for use in communication between the information processing device 1 and the server device 5 encompass public networks, such as the Internet, telephone networks, and satellite communication networks, and leased line networks, such as various local area networks (LANs) including Ethernet (registered trademark), wide area networks (WANs), and internet protocol-virtual private networks (IP-VPNs).

Herein, the information processing device 1 is preferably a terminal device mounted on a head of the user 3. For example, the information processing device 1 may be an ear-hook type terminal, an eyeglass type terminal, or a headband type terminal.

In a case where a person takes exercise, the person unconsciously controls his/her body to reduce horizontal movement of his/her head in order to restrain shaking vision. Therefore, in a case where the information processing device 1 is mounted on the head of the user 3, it is difficult to determine whether or not the user 3 is moving with the use of a dynamic sensor such as an acceleration sensor because the horizontal movement of the head is small. Meanwhile, the information processing device 1 determines whether or not the user 3 is moving with the use of the biological information of the user 3 (more specifically, the heart rate, the blood pressure, the respiratory rate, and the like of the user 3), and therefore it is possible to accurately detect movement of the user 3 even in a case where the information processing device 1 is mounted on the head of the user 3.

Further, in a case where part of or the whole information processing of the information processing device 1 is executed in the server device 5 communicating with the information processing device 1, at least detection of walking or acquisition of biological information is preferably acquired by the various sensors mounted on the head of the user 3.

With the configuration described above, in a case where a moving distance is calculated on the basis of the number of steps, the information processing device 1 according to the present embodiment can exclude a period in which the user 3 is walking without movement (e.g., is stepping on the spot) and only use the number of steps in a period in which the user 3 is walking with movement. Therefore, the information processing device 1 can calculate the moving distance more accurately on the basis of the number of steps.

Further, the information processing device 1 according to the present embodiment can calculate the moving distance on the basis of the number of steps without using positional information obtained by a grobal navigation satellite system (GNSS) represented by a global positioning system (GPS). Therefore, the information processing device 1 does not need to communicate with a GNSS satellite, and thus it is possible to reduce power consumption, as compared to a case where the moving distance is calculated on the basis of positional information of the user 3 acquired by the GNSS or the like. Further, the information processing device 1 can accurately calculate the moving distance even in an environment in which a radio wave from a GNSS satellite is not easily received and positional measurement using a GNSS is difficult (e.g., inside of a building).

[1.2. Functional Configuration of Information Processing Device]

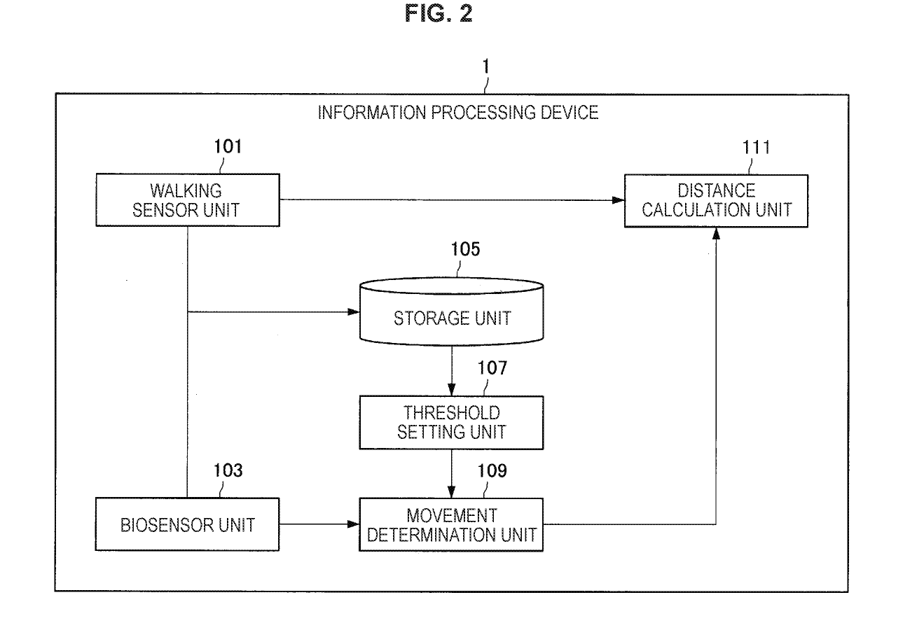

A functional configuration of the information processing device 1 according to the present embodiment will be described with reference to FIG. 2. FIG. 2 is a block diagram showing an internal configuration of the information processing device 1 according to the present embodiment.

As shown in FIG. 2, the information processing device 1 includes a walking sensor unit 101, a biosensor unit 103, a storage unit 105, a threshold setting unit 107, a movement determination unit 109, and a distance calculation unit 111.

The walking sensor unit 101 includes a sensor for detecting walking of the user 3. The walking sensor unit 101 may be made up of, for example, a vibration sensor, an acceleration sensor, or a gyro sensor. Specifically, the vibration sensor detects vibration caused by walking of the user 3 with the use of a pendulum connected to a spring. Further, the acceleration sensor detects a change in speed caused by walking of the user 3 as acceleration. Furthermore, the gyro sensor detects a change in speed caused by walking of the user 3 as an angular velocity. Note that it is needless to say that the walking sensor unit 101 is not limited to the above sensors as long as the walking sensor unit can detect walking of the user 3.

The biosensor unit 103 includes a sensor for detecting biological information of the user 3. Herein, the biological information means information containing at least one of a heart rate, a blood pressure, and a respiratory rate. For example, the biosensor unit 103 is made up of at least one sensor for detecting any one of a heart rate, a blood pressure, and a respiratory rate.

Specifically, the biosensor unit 103 may be a sensor for measuring a heart rate by emitting infrared light to measure a change in blood flow as a change in absorption property of infrared radiation or may be a sensor for measuring a heart rate by detecting pulsation of a blood vessel with the use of a pressure sensor or the like. Further, the biosensor unit 103 may be a sensor for measuring a blood pressure by detecting a change in sound of a blood flow with the use of a microphone or the like or may be a sensor for, by emitting near infrared light, calculating a blood pressure on the basis of a change in amount of blood flow detected on the basis of reflected light. Furthermore, the biosensor unit 103 may be a sensor for detecting respiration with the use of a microphone or the like to measure a respiratory rate. Note that it is needless to say that the biosensor unit 103 is not limited to the above sensors as long as the biosensor unit can detect the heart rate, the blood pressure, or the respiratory rate of the user 3.

The storage unit 105 stores information measured by the walking sensor unit 101 and the biosensor unit 103 in time series. Specifically, the storage unit 105 stores, in time series, a timing of walking of the user 3 detected by the walking sensor unit 101 and the biological information of the user 3 such as the heart rate, the blood pressure, and the respiratory rate measured by the biosensor unit 103. With this, the information processing device 1 can compare time series data of the biological information of the user 3 such as the heart rate, the blood pressure, and the respiratory rate with a timing of walking and therefore extract only the number of steps in a period in which the movement determination unit 109 described later determines that the user is moving.

The movement determination unit 109 determines whether or not the user 3 is moving on the basis of at least one of the heart rate, the blood pressure, and the respiratory rate of the user 3. Specifically, in a case where at least one of the heart rate, the blood pressure, and the respiratory rate of the user 3 is equal to or less than a first threshold thereof, the movement determination unit 109 determines that the user 3 is not moving.

Herein, a load applied to the body of the user 3 in a case where the user 3 is walking without movement (i.e., in a case where the user 3 is stepping on the spot) is lower than a load applied thereto in a case where the user 3 is walking with movement (i.e., in a case where the user 3 is running or walking). Therefore, the movement determination unit 109 can determine whether or not the user is moving with the use of an appropriate threshold (first threshold). Note that the first threshold may be a value set by the threshold setting unit 107 described later on the basis of the past biological information of the user 3 or may be a value input in advance.

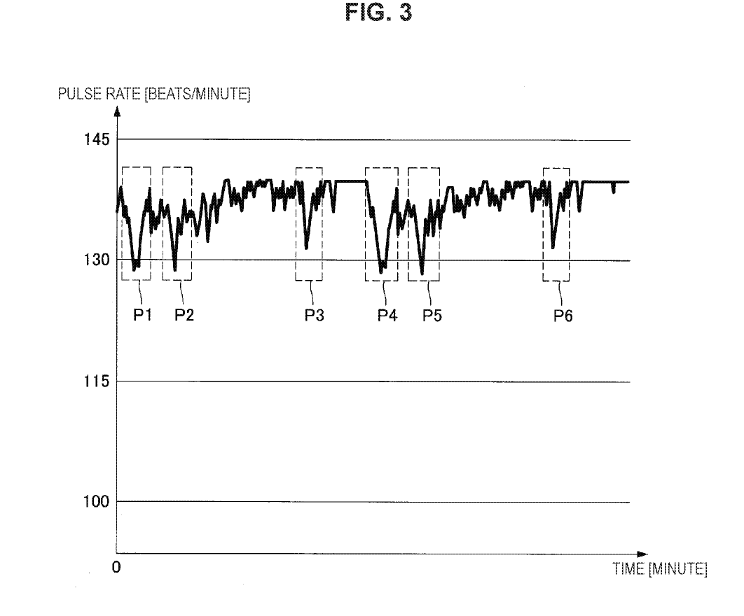

Hereinafter, determination on whether or not a user is moving using the movement determination unit 109 will be described more specifically with reference to FIG. 3. FIG. 3 is a graph showing an example of a temporal change in heart rate obtained in a case where the user 3 is running while stopping occasionally.

As shown in FIG. 3, a heart rate during exercise such as running is higher than a normal heart rate (generally, 60 to 70 beats/minute) and is, for example, approximately 140 beats/minute. Herein, a heart rate in a period (period P1 to period P6) in which the user 3 is not moving and is stepping on the spot because, for example, the user waits for traffic light to change or waits for a crossing to open is approximately 130 to 132 beats/minute and is lower than a heart rate in a period in which the user is moving by running or the like. In view of this, for example, when the first threshold of the heart rate is set to 133 beats/minute, the movement determination unit 109 can determine a period in which the user 3 is not moving and a period in which the user 3 is moving.

Further, the movement determination unit 109 may determine that the user 3 is not moving in a case where a period in which at least one of the heart rate, the blood pressure, and the respiratory rate of the user 3 is equal to or less than the first threshold continues for a predetermined time period.

There is a possibility that, for example, in a case where the user 3 passes a person during running and momentarily stops, the heart rate, the blood pressure, the respiratory rate, and the like are reduced for a short time (e.g., approximately 1 to 3 seconds). The number of steps of the user 3 in such a case is approximately 0 to 2 steps, and therefore it is considered that there is substantially no influence on calculation of a moving distance. In view of this, in a case where the heart rate, the blood pressure, the respiratory rate, and the like are reduced for a short time, the movement determination unit 109 may determine that the user 3 is moving, and, only in a case where the heart rate, the blood pressure, the respiratory rate, and the like are continuously reduced for a predetermined time period, the movement determination unit 109 may determine that the user 3 is not moving. Note that the predetermined time period is changed depending on age, a physical strength, and the like of the user 3 but is approximately, for example, 5 seconds.

With this configuration, the movement determination unit 109 can exclude, from calculation of a moving distance, only a stop for a long time which largely influences the calculation of the moving distance, and therefore it is possible to more simply perform calculation to obtain the moving distance. Further, the movement determination unit 109 can reduce an influence generated when the measured heart rate, blood pressure, respiratory rate, and the like are temporarily reduced due to noise or the like of the biosensor unit 103.

Furthermore, the movement determination unit 109 may determine whether or not the user 3 is moving by combining a plurality of pieces of the biological information of the user 3 acquired by the biosensor unit 103. Specifically, the movement determination unit 109 may determine that the user 3 is not moving in a case where two or more of the heart rate, the blood pressure, and the respiratory rate of the user 3 are equal to or less than the first thresholds thereof. The movement determination unit 109 can determine whether or not the user 3 is moving more accurately by combining a plurality of pieces of the biological information of the user 3.

The threshold setting unit 107 set the first thresholds on the basis of past histories of the heart rate, the blood pressure, the respiratory rate, and the like of the user 3 stored on the storage unit 105. Specifically, the threshold setting unit 107 may calculate a maximum value or average value on the basis of each of the past histories of the heart rate, the blood pressure, the respiratory rate, and the like of the user 3 and set the first threshold on the basis of the calculated maximum value or average value. For example, the threshold setting unit 107 may set a value that is 0.6 to 0.9 times as large as the calculated maximum value as the first threshold. Further, the threshold setting unit 107 may set the calculated average value as the first threshold or may set a value that is 0.6 to 0.9 times as large as the calculated average value as the first threshold.

Furthermore, the threshold setting unit 107 may set a movement average value in a previous history of each of the heart rate, the blood pressure, the respiratory rate, and the like of the user 3 as the first threshold or may set a value that is 0.6 to 0.9 times as large as the movement average value as the first threshold.

The distance calculation unit 111 calculates a moving distance of the user 3 on the basis of the number of steps in a period in which the movement determination unit 109 determines that the user 3 is moving. Specifically, the distance calculation unit 111 calculates a product of the number of steps in the period in which the movement determination unit 109 determines that the user 3 is moving and a step length of the user 3 as the moving distance of the user 3. Herein, the step length of the user 3 may be a value input by the user 3 in advance or may be a value set by the distance calculation unit 111 on the basis of a height, a speed of walking, or the like of the user 3. For example, the step length of the user 3 may be set by the distance calculation unit 111 to a value that is 0.4 to 0.5 times as large as of the height of the user 3.

Further, in a case where the moving distance is calculated in real time while the user 3 is running or walking, the distance calculation unit 111 controls addition of the step length of the user 3 to the moving distance on the basis of whether or not it is determined that the user 3 is moving during detection of walking. Specifically, in a case where it is determined that the user 3 is moving during detection of walking, the distance calculation unit 111 adds the step length of the user 3 to the moving distance, and, in a case where it is determined that the user 3 is not moving, the distance calculation unit 111 does not add the step length of the user 3 to the moving distance.

Note that, in a case where the movement determination unit 109 cannot accurately determine whether or not the user 3 is moving, the distance calculation unit 111 may calculate the moving distance of the user 3 on the basis of positional information of the user 3. Specifically, in a case where at least one of the heart rate, the blood pressure, and the respiratory rate of the user 3 is equal to or less than a second threshold smaller than the first threshold, the distance calculation unit 111 may calculate the moving distance of the user 3 on the basis of the positional information of the user 3.

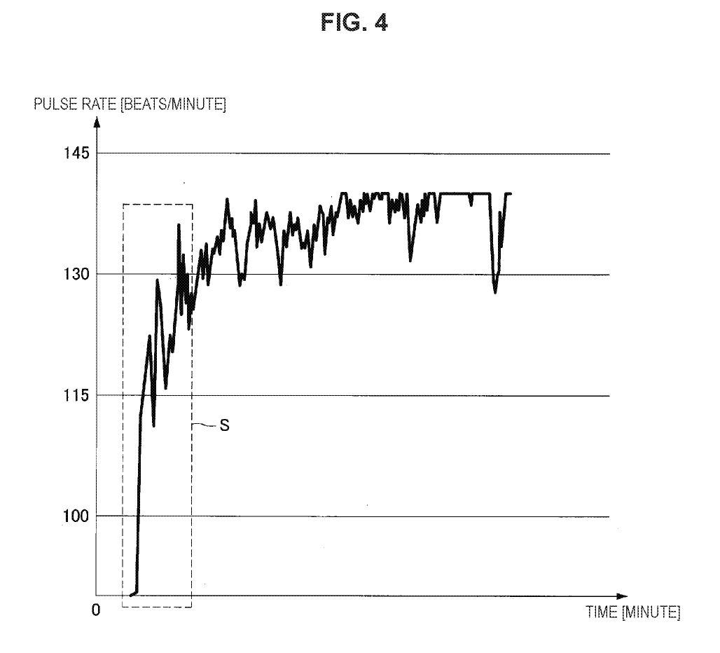

Such a case will be described with reference to FIG. 4. FIG. 4 is a graph showing an example of a temporal change in heart rate immediately after the user 3 starts running.

As shown in FIG. 4, in a period (period S) immediately after the start of exercise such as running, the heart rate is gradually increased from a normal heart rate and then reaches a heart rate during exercise (e.g., approximately 140 beats/minute). Therefore, in a case where a value set on the basis of the heart rate during exercise is used as the first threshold, there is a possibility that the movement determination unit 109 cannot determine stepping of the user 3 without movement in the period S.

In view of this, the distance calculation unit 111 may determine that the heart rate of the user 3 does not reach the heart rate during exercise such as running in a case where at least one of the heart rate, the blood pressure, and the respiratory rate of the user 3 is equal to or less than the second threshold thereof.

In such a case, the distance calculation unit 111 may calculate the moving distance of the user 3 on the basis of positional information of the user 3 acquired by a GNSS or the like. Specifically, the distance calculation unit 111 may acquire positional information of the user 3 as necessary with the use of a GNSS or the like, set a line segment connecting positions of the user 3 at respective points of time as a path through which the user 3 has moved, and calculate a distance of the path as a moving distance of the user 3.

Note that the second threshold is set by the threshold setting unit 107 as a value smaller than the first threshold. Herein, the second threshold, as well as the first threshold, may be set on the basis of a maximum value or average value of each of the past histories of the heart rate, the blood pressure, the respiratory rate, and the like of the user 3.

With the configuration described above, the information processing device 1 according to the present embodiment can accurately calculate the moving distance of the user 3 on the basis of the number of steps.

[1.3. Modification Example of Information Processing Device]

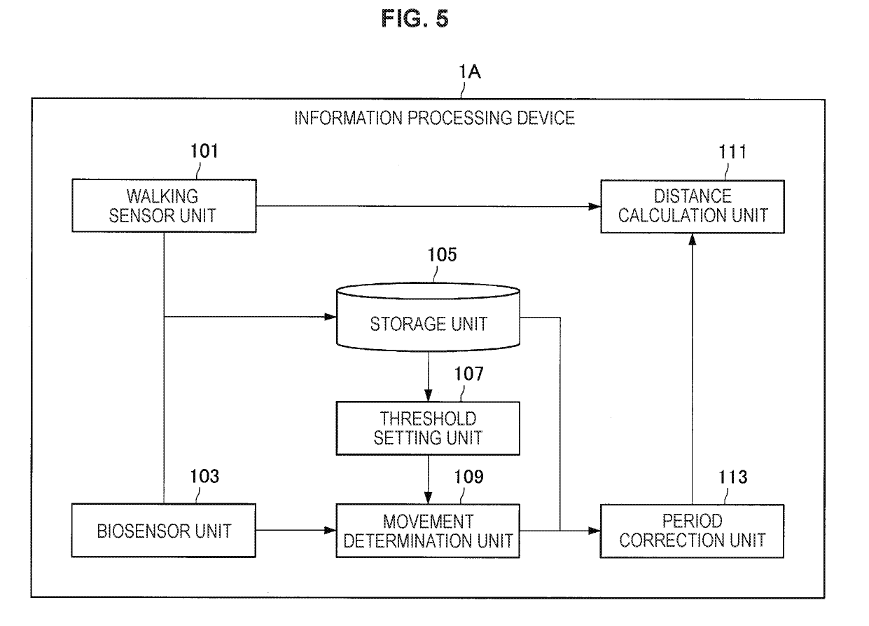

A modification example of the information processing device according to the present embodiment will be described with reference to FIG. 5. FIG. 5 is a block diagram showing an internal configuration of an information processing device 1A according to a modification example of the present embodiment.

As shown in FIG. 5, the information processing device 1A according to the modification example is different from the information processing device 1 shown in FIG. 2 in that the information processing device 1A further includes a period correction unit 113. Note that the walking sensor unit 101, the biosensor unit 103, the storage unit 105, the threshold setting unit 107, the movement determination unit 109, and the distance calculation unit 111 are substantially similar to those of the information processing device 1, and therefore description thereof is herein omitted. Hereinafter, the period correction unit 113 that is a feature of the information processing device 1A according to the modification example will be described.

The period correction unit 113 corrects a start point and an end point of a period in which the movement determination unit 109 determines that the user 3 is not moving. Specifically, the period correction unit 113 performs correction to delay the start point and the end point of the period in which it is determined that the user 3 is not moving. This is because reduction in the heart rate, the blood pressure, and the respiratory rate of the user 3 caused by a stop of movement of the user 3 may be delayed from the stop of movement of the user 3. The period correction unit 113 can more accurately specify the period in which it is determined that the user 3 is not moving by correcting a start point and an end point of a period in which the heart rate, the blood pressure, and the respiratory rate of the user 3 are reduced.

Note that the period correction unit 113 may correct the start point and the end point of the period in which it is determined that the user 3 is not moving with the use of different amounts of correction or may correct one of the start point and the end point. The amounts of correction of the start point and the end point using the period correction unit 113 may be set on the basis of, for example, age, an amount of exercise, and a frequency of exercise of the user 3, a history of biological information of the user 3 stored on the storage unit 105, and the like.

According to the information processing device 1A according to the modification example of the present embodiment, it is possible to further accurately calculate a moving distance on the basis of the number of steps.

In the above description, the functional configurations of the information processing devices according to the present embodiment and the modification example have been described in detail.

[1.4. Control of Information Processing Device]

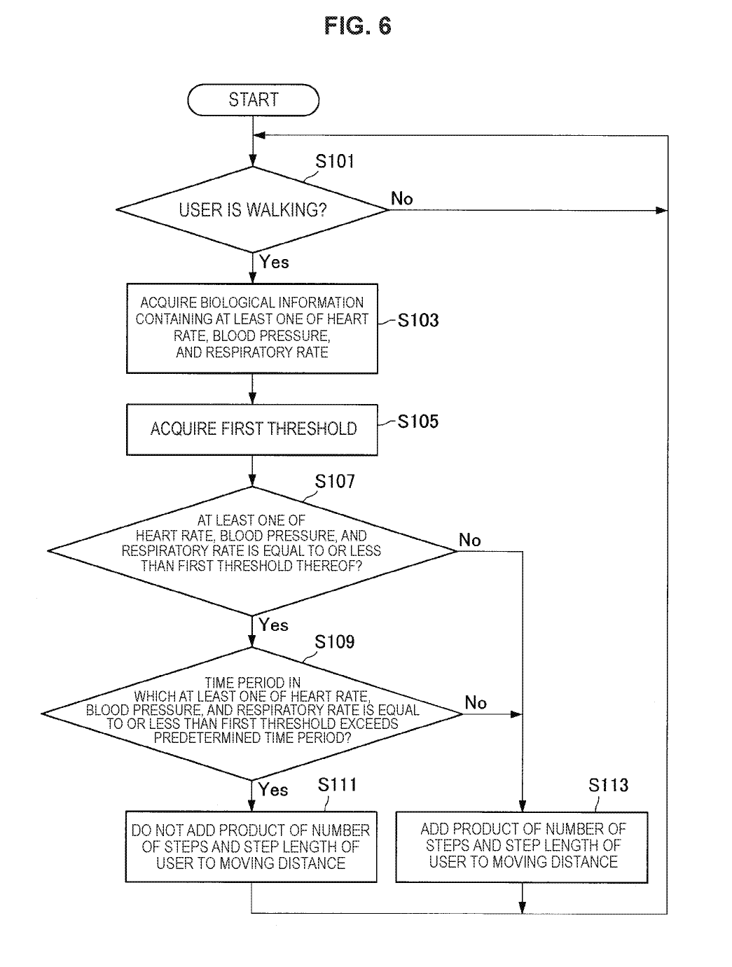

A control example of the information processing device 1 according to the present embodiment will be described with reference to FIG. 6. FIG. 6 is a flowchart showing an example of control operation executed by the information processing device 1 according to the present embodiment.

As shown in FIG. 6, first, the walking sensor unit 101 detects whether or not the user 3 is walking (S101). Note that “walking” herein includes a case where the user 3 is walking and, in addition, a case where the user 3 is jogging or running. In a case where walking of the user 3 is not detected (S101/No), the walking sensor unit 101 waits until the walking sensor unit 101 detects walking of the user 3. On the contrary, in a case where walking of the user 3 is detected (S101/Yes), the movement determination unit 109 acquires biological information containing at least one of the heart rate, the blood pressure, and the respiratory rate of the user 3 measured by the biosensor unit 103 (S103). Further, the movement determination unit 109 acquires the first threshold from the threshold setting unit 107 (S105).

Herein, the movement determination unit 109 determines whether or not the heart rate, the blood pressure, and the respiratory rate are equal to or less than the respective first thresholds (S107). In a case where any one of the heart rate, the blood pressure, and the respiratory rate is equal to or less than the first threshold thereof (S107/Yes), the movement determination unit 109 further determines whether or not a time period in which any one of the heart rate, the blood pressure, and the respiratory rate is equal to or less than the first threshold exceeds a predetermined time period (S109). In a case where the time period in which any one of the heart rate, the blood pressure, and the respiratory rate is equal to or less than the first threshold exceeds the predetermined time period in S109 (S109/Yes), the distance calculation unit 111 does not add a product of the number of steps and a step length of the user 3 to a moving distance (S111).

On the contrary, in a case where the heart rate, the blood pressure, and the respiratory rate are more than the respective first thresholds in S107 (S107/No), the distance calculation unit 111 adds the step length of the user to the moving distance (S113). Further, in a case where the time period in which any one of the heart rate, the blood pressure, and the respiratory rate is equal to or less than the first threshold is equal to or less than the predetermined time period in S109 (S109/Yes), the distance calculation unit 111 adds the product of the number of steps and the step length of the user 3 to the moving distance (S113).

After S111 and S113, the information processing device 1 returns to S101 and repeats the control operation. With this, the information processing device 1 can calculate the moving distance of the user 3 on the basis of the number of steps.

Further, the movement determination unit 109 may acquire time series data of the number of steps and biological information of the user 3 and then specify a period in which the user 3 is walking with movement on the basis of the heart rate, the blood pressure, and the respiratory rate of the user 3. Specifically, the movement determination unit 109 determines a period in which the heart rate, the blood pressure, and the respiratory rate of the user 3 are more than the first thresholds and specifies the period as a period in which the user is moving. Further, the distance calculation unit 111 calculates a moving distance of the user 3 on the basis of the number of steps in the period in which it is determined that the user is moving. In such a case, the period correction unit 113 may perform correction to delay a start point and an end point of the period in which it is determined that the user is moving.

### 2. HARDWARE CONFIGURATION

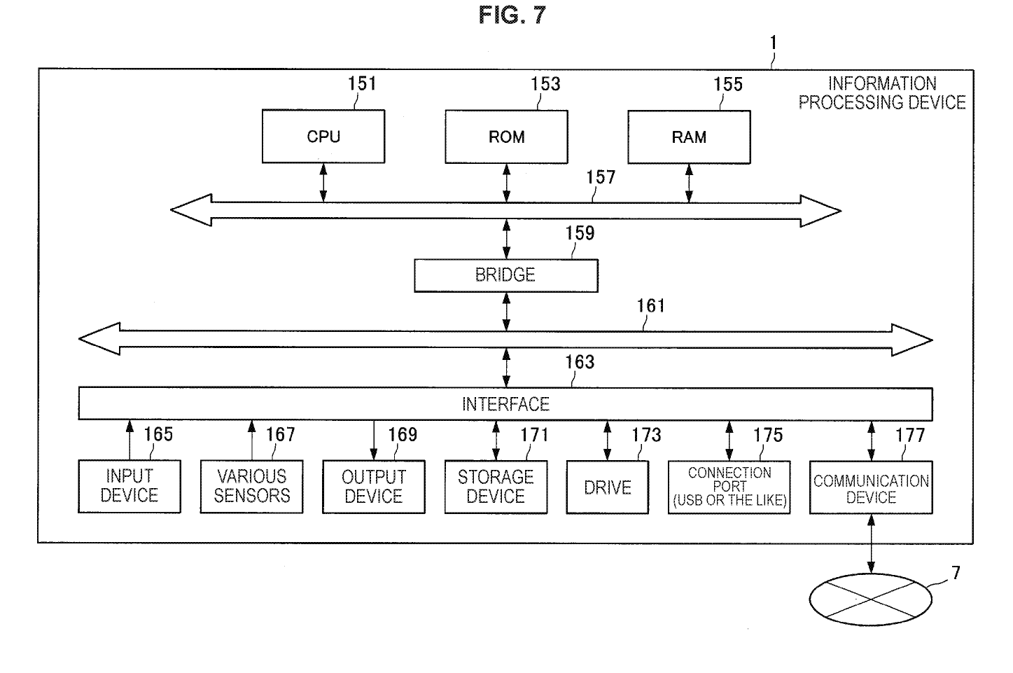

A hardware configuration of the information processing device 1 according to the present embodiment will be described with reference to FIG. 7. FIG. 7 is a block diagram showing an example of a hardware configuration of the information processing device 1 according to the present embodiment. Note that information processing according to the present embodiment is realized by cooperation of software and hardware.

As shown in FIG. 7, the information processing device 1 includes a central processing unit (CPU) 151, a read only memory (ROM) 153, a random access memory (RAM) 155, a bridge 159, internal buses 157 and 161, an interface 163, an input device 165, and various sensors 167, an output device 169, a storage device 171, a drive 173, a connection port 175, and a communication device 177.

The CPU 151 functions as an arithmetic processing unit and a control device and controls the whole operation in the information processing device 1 in accordance with a program according to the present embodiment. The ROM 153 stores a program and an operation parameter used by the CPU 151, and the RAM 155 temporarily stores a program for use in execution of the CPU 151, a parameter appropriately changed in the execution, and the like. The CPU 151 executes functions of, for example, the threshold setting unit 107, the movement determination unit 109, the distance calculation unit 111, and the period correction unit 113.

Those CPU 151, ROM 153, and RAM 155 are connected to one another via the bridge 159, the internal buses 157 and 161, and the like. The CPU 151, the ROM 153, and the RAM 155 are also connected to the input device 165, the various sensors 167, the output device 169, the storage device 171, the drive 173, the connection port 175, and the communication device 177 via the interface 163.

The input device 165 is made up of, for example, an input device via which the user 3 inputs information, such as a touchscreen, a keyboard, a button, a microphone, a switch, and a lever, and an input control circuit for generating an input signal on the basis of input from the user and outputting the input signal to the CPU 151.

The various sensors 167 include, for example, a vibration sensor, an acceleration sensor, and a gyro sensor for detecting walking of the user 3. Further, the various sensors 167 include, for example, a heart rate meter, a sphygmomanometer, and a respiratory rate meter for measuring biological information of the user 3. Further, the various sensors 167 may include a GNSS sensor, a geomagnetic sensor, a barometric sensor, a temperature sensor, and the like. The various sensors 167 execute functions of, for example, the walking sensor unit 101, the biosensor unit 103, and the like.

The output device 169 includes, for example, a display device such as a liquid crystal display (LCD) device, an organic electroluminescence display (OLED) device, and a lamp. The output device 169 may further include a sound output device such as a speaker and a headphone. For example, the display device displays a generated image, and the sound output device converts sound data or the like into sound and outputs the sound.

The storage device 171 is a device for storing data configured as an example of a storage unit of the information processing device 1. The storage device 171 may include a storage medium, a storage device for storing data on the storage medium, a reading device for reading data from the storage medium, and a deleting device for deleting the stored data. The storage device 171 executes, for example, a function of the storage unit 105.

The drive 173 is a reader/writer for a storage medium and is provided inside the information processing device 1 or is externally attached thereto. The drive 173 reads information stored on a removable storage medium such as an attached semiconductor memory and outputs the information to the RAM 155. Further, the drive 173 can also write information to the removable storage medium.

The connection port 175 is a connection interface made up of, for example, a connection port for connecting an external connection apparatus, such as a universal serial bus (USB) port or an optical audio terminal.

The communication device 177 is, for example, a communication interface made up of, for example, a communication device to be connected to a network 7 such as a public network or a leased line network. Further, the communication device 177 may be a communication device compliant with a wireless LAN or may be a cable communication device that performs cable communication via a wired network.

It is also possible to prepare a computer program for causing hardware such as a CPU, a ROM, and a RAM included in the information processing device 1 to have functions equal to those of the respective configurations of the information processing device 1 according to the present embodiment described above. Further, a storage medium storing the computer program is also provided.

### 3. CONCLUSION

As described above, the information processing device 1 according to the present embodiment can calculate a moving distance on the basis of the number of steps without using positional information obtained by a GNSS or the like. Therefore, the information processing device 1 can reduce power consumption, as compared to a case where the moving distance is calculated on the basis of the positional information of the user 3 acquired by the GNSS or the like. Further, the information processing device 1 can accurately calculate a moving distance even in an environment in which a radio wave from a GNSS satellite is not easily received and positional measurement using a GNSS is difficult (e.g., inside of a building).

Further, the information processing device 1 according to the present embodiment determines whether or not the user 3 is moving on the basis of biological information of the user 3 such as the heart rate, the blood pressure, and the respiratory rate. Therefore, even in a case where it is difficult to determine whether or not the user 3 is moving with the use of a dynamic sensor, the information processing device 1 can accurately determine whether or not the user 3 is moving.

Therefore, the information processing device 1 can calculate a moving distance of the user 3 on the basis of the number of steps in a period in which it is determined that the user 3 is moving, and therefore it is possible to accurately calculate the moving distance.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art based on the description of this specification.

Additionally, the present technology may also be configured as below.

(1)

An information processing device, including:

a movement determination unit configured to determine whether or not a user is moving on the basis of at least one of a heart rate, a blood pressure, and a respiratory rate of the user; and

a distance calculation unit configured to calculate a moving distance of the user on the basis of the number of steps of the user in a period in which the movement determination unit determines that the user is moving.

(2)

The information processing device according to (1),

wherein, in a case where at least one of the heart rate, the blood pressure, and the respiratory rate of the user is equal to or less than a first threshold, the movement determination unit determines that the user is not moving.

(3)

The information processing device according to (2),

wherein the first threshold is a value set on the basis of each of past histories of the heart rate, the blood pressure, and the respiratory rate of the user.

(4)

The information processing device according to (2) or (3),

wherein the first threshold is a value set on the basis of a past maximum value or average value of each of the heart rate, the blood pressure, and the respiratory rate of the user.

(5)

The information processing device according to any one of (2) to (4),

wherein, in a case where a time period in which at least one of the heart rate, the blood pressure, and the respiratory rate of the user is equal to or less than the first threshold is less than a predetermined time period, the movement determination unit determines that the user is moving.

(6)

The information processing device according to any one of (2) to (5),

wherein, in a case where at least one of the heart rate, the blood pressure, and the respiratory rate of the user is equal to or less than a second threshold that is less than the first threshold, the distance calculation unit calculates the moving distance of the user on the basis of positional information of the user.

(7)

The information processing device according to any one of (1) to (6), further including:

a period correction unit configured to correct a start point and an end point of a period in which the movement determination unit determines that the user is not moving.

(8)

The information processing device according to any one of (1) to (7),

wherein the distance calculation unit calculates a product of the number of steps of the user and a step length of the user as the moving distance of the user.

(9)

The information processing device according to any one of (1) to (8),

wherein at least one of the number of steps, the heart rate, the blood pressure, and the respiratory rate of the user is acquired by a sensor mounted on a head of the user.

(10)

An information processing method, including:

determining whether or not a user is moving on the basis of at least one of a heart rate, a blood pressure, and a respiratory rate of the user; and

calculating a moving distance of the user on the basis of the number of steps of the user in a period in which a central processing unit determines that the user is moving.

(11)

A program causing a computer to function as:

a movement determination unit configured to determine whether or not a user is moving on the basis of at least one of a heart rate, a blood pressure, and a respiratory rate of the user; and

a distance calculation unit configured to calculate a moving distance of the user on the basis of the number of steps of the user in a period in which the movement determination unit determines that the user is moving.

### REFERENCE SIGNS LIST

1, 1A information processing device
3 user
5 server device
101 walking sensor unit
103 biosensor unit
105 storage unit
107 threshold setting unit
109 movement determination unit
111 distance calculation unit
113 period correction unit

1. An information processing device, comprising:
a movement determination unit configured to determine whether or not a user is moving on the basis of at least one of a heart rate, a blood pressure, and a respiratory rate of the user; and
a distance calculation unit configured to calculate a moving distance of the user on the basis of the number of steps of the user in a period in which the movement determination unit determines that the user is moving.

2. The information processing device according to claim 1,
wherein, in a case where at least one of the heart rate, the blood pressure, and the respiratory rate of the user is equal to or less than a first threshold, the movement determination unit determines that the user is not moving.

3. The information processing device according to claim 2,
wherein the first threshold is a value set on the basis of each of past histories of the heart rate, the blood pressure, and the respiratory rate of the user.

4. The information processing device according to claim 3,
wherein the first threshold is a value set on the basis of a past maximum value or average value of each of the heart rate, the blood pressure, and the respiratory rate of the user.

5. The information processing device according to claim 2,
wherein, in a case where a time period in which at least one of the heart rate, the blood pressure, and the respiratory rate of the user is equal to or less than the first threshold is less than a predetermined time period, the movement determination unit determines that the user is moving.

6. The information processing device according to claim 2,
wherein, in a case where at least one of the heart rate, the blood pressure, and the respiratory rate of the user is equal to or less than a second threshold that is less than the first threshold, the distance calculation unit calculates the moving distance of the user on the basis of positional information of the user.

7. The information processing device according to claim 1, further comprising:
a period correction unit configured to correct a start point and an end point of a period in which the movement determination unit determines that the user is not moving.

8. The information processing device according to claim 1,
wherein the distance calculation unit calculates a product of the number of steps of the user and a step length of the user as the moving distance of the user.

9. The information processing device according to claim 1,
wherein at least one of the number of steps, the heart rate, the blood pressure, and the respiratory rate of the user is acquired by a sensor mounted on a head of the user.

10. An information processing method, comprising:
determining whether or not a user is moving on the basis of at least one of a heart rate, a blood pressure, and a respiratory rate of the user; and
calculating a moving distance of the user on the basis of the number of steps of the user in a period in which a central processing unit determines that the user is moving.

11. A program causing a computer to function as:
a movement determination unit configured to determine whether or not a user is moving on the basis of at least one of a heart rate, a blood pressure, and a respiratory rate of the user; and
a distance calculation unit configured to calculate a moving distance of the user on the basis of the number of steps of the user in a period in which the movement determination unit determines that the user is moving.
